(19) United States
(12) Patent Application Publication
(10) Pub. No.: US 2024/0130652 A1
(43) Pub. Date: Apr. 25, 2024

(54) SYSTEMS, APPARATUS, AND METHODS FOR ASSESSING COGNITIVE DECLINE BASED UPON MONITORED DRIVING PERFORMANCE

(71) Applicant: BOARD OF TRUSTEES OF MICHIGAN STATE UNIVERSITY, East Lansing, MI (US)

(72) Inventors: Zhaojian Li, Ann Arbor, MI (US); Wen Li, Okemos, MI (US); Michele Grimn, Canton, MI (US)

(21) Appl. No.: 18/278,441

(22) PCT Filed: Feb. 18, 2022
(86) PCT No.: PCT/US22/16900

§ 371 Date: Aug. 23, 2023

Related U.S. Application Data
(60) Provisional application No. 63/152,604, filed on Feb. 23, 2021.

(51) Int. Cl. A61B 5/18; A61B 5/00; A61B 5/0205

(52) U.S. Cl. CPC A61B 5/18; A61B 5/0205; A61B 5/4088; A61B 5/4842; A61B 5/6893; A61B 5/7267; A61B 5/0531

(57) ABSTRACT

Example systems, apparatus, and methods for assessing cognitive decline based upon monitored driving performance are disclosed. An example system includes a sensor suite configured to be used in conjunction with a vehicle, and a computing device. The sensor suite comprising one or more sensors configured to sense one or more physiological signals of a driver while the driver grips the steering wheel and operates the vehicle, one or more converters configured to convert the one or more sensed physiological signals into physiological data representative of one or more physiological states, and an imaging device configured to capture image data representative of the vehicle within an environment of use. The computing device comprising one or more processors configured to receive the physiological data and the image data from the sensor suite and determine one or more biomarkers representative of cognitive decline based upon the physiological data and the image data.

### CROSS-REFERENCE TO RELATED APPLICATION

This application claims the benefit of U.S. Provisional Patent Application No. 63/152,604, entitled “Systems and Methods for Monitoring Driving Performance,” and filed on Feb. 23, 2021. U.S. Provisional Patent Application No. 63/152,604 is hereby incorporated herein by reference in its entirety. Priority to U.S. Provisional Patent Application No. 63/152,604 is hereby claimed.

### FIELD OF THE DISCLOSURE

This disclosure relates generally to assessing cognitive decline, and, more particularly, to systems, apparatus, and methods for assessing cognitive decline based upon paired physiological measurements and monitored driving performance.

### BACKGROUND

Cognitive decline is common within a significant portion of the aging population. In the United States (US) in 2017, there were approximately 6 million individuals over the age of 65 with clinical Alzheimer's disease (AD) or mild cognitive impairment due to AD. In addition, there were almost 47 million individuals with preclinical AD. As the percentage of the population over the age of 65 increases, it is expected that the number of individuals in the US with AD will increase to 15 million by 2060. The cognitive changes that result from AD and other causes of dementia, such as vascular dementia, are typically gradual and continuous in nature. Accurate assessment of cognitive decline requires longitudinal assessment in order to determine the change(s) with respect to a prior, normal level of mental acuity. Such changes can be used to identify individuals who may need specialized care as well as individuals who likely have an ability to live independently. Such assessments can help ensure the safety of individuals with cognitive decline and enable those unaffected to safely continue their normal activities.

### SUMMARY

The disclosure generally relates to a multi-modality driver assessment system that is capable of continuously and unobtrusively monitoring a driver while they are driving to assess the driver's driving behaviors, driving conditions, and associated physiological states to assess the health and/or cognitive ability of the driver. The driver assessment system includes a monitoring system implemented in conjunction with a motor vehicle. The monitoring system includes an add-on smart steering wheel sleeve or cover that includes one or more embedded physiological sensors and is adapted to be secured to a steering wheel of the vehicle. When the driver's hands come into contact with the physiological sensors, by gripping the steering wheel to operate the vehicle, the physiological sensors sense one or more physiological signals of the driver as they operate the vehicle. Example physiological sensors include an electromyography (EMG) sensor, a heart rate or pulse sensor, a gripping force sensor, and an electrodermal activity (EDA) sensor. Such sensors can be used to sense and measure physiological states of the driver that may, for example, be indicative of stress or other conditions of the driver under different driving conditions which, in turn, can be indicative of cognitive decline. The driver monitoring system also includes an imaging device to capture image data representative of the vehicle within an environment of use. The image data can be processed to determine driving behaviors and/or driving conditions that can provide contextual information for identified physiological states within a cognition-involved driving environment. Example driving behaviors include occurrences of driving lane deviations, maintenance of appropriate inter-vehicle distances, and missing stop signs, etc. Example driving conditions include daytime, nighttime, rain, snow, wind, wet pavement, and icy pavement.

Sensed physiological data and captured image data are provided to a server for processing to determine one or more biomarkers representative of cognitive decline. In some examples, image data is processed with one or more computer vision algorithms to detect driving behaviors and/or driving conditions. Additionally and/or alternatively, one or more trained machine learning models can be used to process image data to detect driving behaviors and/or driving conditions. The detected driving behaviors and/or driving conditions, and temporally associated physiological data can be processed to determine the one or more biomarkers. In some examples, the detected driving behaviors and/or driving conditions, along with the physiological data, are inputs to one or more trained machine learning models to determine the one or more biomarkers representative of cognitive decline. Accordingly, disclosed examples provide a non-intrusive, inexpensive, and convenient way to longitudinally monitor drivers' cognitive decline and/or to provide an objective driving capability assessment that can be very helpful for drivers and/or their caregivers when deciding whether to cease driving.

While examples are described with reference to assessing cognitive decline, persons of ordinary skill in the art will recognize that disclosed examples can additionally and/or alternatively be used to make other health, ability, and/or cognitive assessments. Other example assessments include driver impairment detection (e.g., due to alcohol, drug, fatigue, etc.), driver health monitoring, disease diagnosis, disease prognosis, driving performance monitoring (e.g., for aged populations and novice drivers), and vehicle fleet safety management.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate examples of concepts that include the claimed invention(s) and explain various principles and advantages of those examples.

FIG. 1 is a block diagram of an example driver assessment system, in accordance with the disclosure.

FIG. 2 is a block diagram of an example monitoring system including a sensor suite that can be used to implement the example monitoring systems of FIG. 1, in accordance with the disclosure.

FIG. 3A is a top view of a portion of an example sensor suite, in accordance with the disclosure.

FIG. 3B is cross section view of the example sensor suite of FIG. 3A, in accordance with the disclosure.

FIG. 4A is a top view of a portion of another example sensor suite, in accordance with the disclosure.

FIG. 4B is cross section view of the example sensor suite of FIG. 4A, in accordance with the disclosure.

FIG. 5A is a top view of a portion of yet another example sensor suite, in accordance with the disclosure.

FIG. 5B is cross section view of the example sensor suite of FIG. 5A, in accordance with the disclosure.

FIG. 6 is a schematic diagram of example analog circuit configured to be used to determine a skin impedance, in accordance with the disclosure.

FIG. 7 is a schematic diagram of example analog circuit configured to be used to determine a gripping force, in accordance with the disclosure.

FIG. 8 is a block diagram of an example implementation of the example cognitive ability analyzer of FIG. 1, in accordance with the disclosure.

FIG. 9 is a block diagram of an example machine learning framework that can be used to implement the example cognitive ability analyzers of FIGS. 1 and 8, in accordance with the disclosure.

FIG. 10 is a flowchart representative of an example method, hardware logic, machine-readable instructions, or software for assessing cognitive ability, in accordance with the disclosure.

FIG. 11 is a block diagram of an example logic circuit for implementing example methods and/or operations described herein.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of examples of the disclosure.

The system, apparatus, and method components have been represented where appropriate by conventional symbols in the drawings, showing only those specific details that are pertinent to understanding the examples of the disclosure so as not to obscure the disclosure with details that will be readily apparent to those of ordinary skill in the art having the benefit of the description herein.

The figures are not to scale. Instead, to clarify multiple layers and regions, the thickness of the layers may be enlarged in the drawings. As used in this patent, stating that any part (e.g., a layer, film, area, region, or plate) is in any way on (e.g., positioned on, located on, disposed on, or formed on, etc.) another part, indicates that the referenced part is either in contact with the other part, or that the referenced part is above the other part with one or more intermediate part(s) located therebetween. Stating that any part is in contact with another part means that there is no intermediate part between the two parts. Although the figures show layers and regions with clean lines and boundaries, some or all of these lines and/or boundaries may be idealized. In reality, the boundaries and/or lines may be unobservable, blended, and/or irregular. Use of terms such as up, down, top, bottom, side, end, front, back, etc. herein are used with reference to a currently considered or illustrated orientation. If they are considered with respect to another orientation, it should be understood that such terms must be correspondingly modified.

### DETAILED DESCRIPTION

Highly specific biomarkers of cognitive decline can be obtained through positron emission tomography (PET) imaging, lumbar puncture, etc. However, such procedures are invasive, expensive, and not appropriate for ongoing or regular monitoring over time. Semi-objective tracking of cognitive changes can be obtained through sequential application of tools such as the Mini-Mental Status Exam (MMSE). However, doing so on a regular basis is not feasible. Moreover, clinical assessments can be affected by inter-assessor variability.

It has been advantageously discovered that physiological indicators can be indicative of changes or decline in cognitive ability, especially if sensed and measured during everyday activities that require complex cognitive activity and/or cognitive integration. However, traditional methods of monitoring a person during everyday activities have been plagued with limitations including, but not limited to, the feasibility of deploying sensors outside of a research environment and the requirement that a person make an active decision to wear a sensor. Accordingly, there is a need for systems, apparatus, and methods that can unobtrusively and continuously record physiological indicators during cognitively-complex activities.

An example cognitively-complex activity is operating a motor vehicle. Driving is an activity known to be affected by acute and chronic changes in cognitive ability. Changes in driving ability have been linked to changes in both low-level functions such as attention and perception, and high-level executive functions such as inhibition. Driving is for many individuals a key part of maintaining independence, and driving cessation has been associated with increased morbidity among individuals—including depression, decline in health, and reduction of engagement with the community, to name some. For some individuals who are anxious about early signs of cognitive decline, giving up driving earlier than necessary may limit their independence and have broader negative impacts. For others who insist on driving despite cognitive decline, there may be a time when their reduced driving ability creates a safety hazard for themselves and/or others. The ability to safely operate a vehicle includes a variety of skills, such as an ability to stay within the intended lane, a sufficiently short reaction time when exposed to an unexpected hazard (such as an object in the road or a stopped vehicle), the ability to maintain a speed that fits within the expected range of current traffic (i.e., not too fast, but not too slow), the ability to judge driving conditions, etc.

Physiological indicators, such as heart rate, electrodermal activity (EDA) (e.g., sweatiness of palms), electromyography (EMG) activity, and gripping force applied to a steering wheel, are not themselves an indicator of cognitive decline or impairment. However, it has been advantageously discovered that such physiological indicators, when correlated with specific driving behaviors and/or specific driving conditions, can be indicative of decreased driving ability and/or decreased cognitive ability.

Accordingly, there is a need for systems, apparatus, and methods that can unobtrusively and continuously record physiological indicators while a driver operates a motor vehicle, and to process such physiological indicators in conjunction with detected driving behaviors and/or driving conditions to assess cognitive decline.

Reference will now be made in detail to non-limiting examples, some of which are illustrated in the accompanying drawings.

### Example Driver Assessment Systems

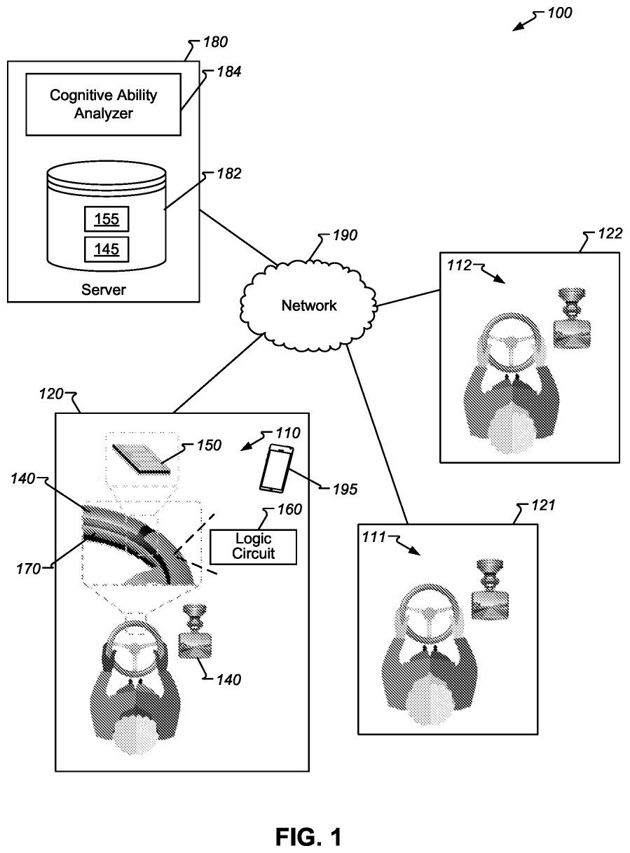

FIG. 1 is a block diagram of an example multi-modality driver assessment system 100 that can be used to monitor drivers while they are operating motor vehicles (e.g., driving, parking, etc.) to assess the drivers' driving behaviors and temporally associated physiological states to assess the health and/or cognitive abilities of the drivers. Example motor vehicles includes cars, vans, trucks, and motorcycles.

The example driver assessment system 100 includes one or more multi-modality monitoring systems (three of which are designated at reference numerals 110, 111, and 112) implemented in conjunction with respective ones of one or more motor vehicles (three of which are designated at reference numerals 120, 121, and 122) to continuously and unobtrusively sense, measure, capture, and record physiological data representative of physiological states, driving behaviors, and/or driving conditions associated with operations of the motor vehicles 120-122.

The example monitoring system 110 implemented in conjunction with the vehicle 120 includes an add-on smart steering wheel sleeve or cover 130 and an imaging device 140. The steering wheel sleeve or cover 130 includes one or more embedded physiological sensors 150 and a logic circuit 160, and it is adapted to be secured to a steering wheel 170 of the vehicle 120. In another example, the physiological sensors 150 and logic circuit 160 are directly secured (e.g., affixed or adhered) to the steering wheel 170. Example imaging devices 140 include a still picture camera, a video camera, and a combination of both. While not shown in FIG. 1 for clarity of illustration, the imaging device 140 can be mounted to a dashboard and/or other location of the vehicle 120 such that it can capture, while the vehicle 120 is being operated, image data 145 representing relationships between the vehicle 120 and an environment in which the vehicle 120 is operating and/or driving conditions. For example, the image data 145 can include one or more of front, side, and rear views of the environment relative to the vehicle 120. While not shown for clarity of illustration, the other monitoring systems 111 and 112 can be similarly implemented.

When a driver grips the steering wheel 170 to operate the vehicle 120, their hands come into physical and/or electrical contact with the physiological sensors 150, and the physiological sensors 150 can continuously and unobtrusively sense one or more physiological signals of the driver as they operate the vehicle 120.

The logic circuit 160 is configured to convert or transform the physiological signals sensed by the sensors 150 into physiological data 155 representative of physiological states of the driver. The physiological states can, for example, be indicative of stress or another condition of the driver during different driving situations and/or behaviors, and/or under different driving conditions that, in turn, can be indicative of cognitive decline. Example physiological sensors include an EMG sensor, a heart rate or pulse sensor, a gripping force sensor, and an EDA sensor.

The logic circuit 160 is configured to communicate, transmit, or otherwise convey the image data 145 and the physiological data 155 to an example server 180 (e.g., via a suitable wireless communication network or protocol) for processing to determine one or more biomarkers representative of cognitive decline. Additionally and/or alternatively, the imaging device 140, rather than the logic circuit 160, conveys the image data 145 to the server 180. In some examples, the logic circuit 160 communicates the image data 145 and the physiological data 155 to the server 180 via a network 190, such as The Internet. For example, the logic circuit 160 can communicate the image data 145 and the physiological data 155 directly to the server 180, and/or via a Bluetooth® interface or a universal serial bus (USB) interface to a nearby computing device 195 (e.g., a mobile phone, or tablet). The computing device 195 can, in turn, communicate the image data 145 and the physiological data 155 to the server 180. In some examples, the logic circuit 160 streams the image data 145 and the physiological data 155 to the server 180 as it is captured. However, the image data 145 and the physiological data 155 can be temporarily stored and/or aggregated before being conveyed to the server 180. Additionally and/or alternatively, the logic circuit 160 can store the image data 145 and the physiological data 155 on a removable storage medium, such as a flash drive, or memory card for subsequent retrieval.

The example server 180 includes one or more tangible or non-transitory storage devices 182 to store the image data 145 and the physiological data 155. Example storage devices 182 include a hard disk drive, a digital versatile disk (DVD), a compact disc (CD), a solid-state drive (SSD), flash memory, read-only memory, and random-access memory. The image data 145 and the physiological data 155 data can be stored for any suitable duration of time (e.g., permanently, for an extended period of time (e.g., while a program associated with the machine-readable instructions is executing), and/or a short period of time (e.g., while the machine-readable instructions are cached and/or during a buffering process)).

The example server 180 includes an example cognitive ability analyzer 184 configured to process the image data 145 and the physiological data 155 to determine one or more biomarkers representative of cognitive decline. The cognitive ability analyzer 184 processes the image data 145 to determine driving behaviors and/or driving conditions that can provide contextual information for associated detected physiological states. Example driving behaviors include an ability to stay within an intended lane (e.g., lane deviations), maintain inter-vehicle distances, demonstrate an appropriate reaction time when exposed to an unexpected hazard or driving condition (such as an object in the road or a stopped vehicle), an ability to maintain a speed that fits within the expected range of current traffic (e.g., not too fast, but not too slow), identify and respond appropriately to stop signs, and/or any other driving behaviors. The cognitive ability analyzer 184 can also process the image data 145 to detect driving conditions, such as daytime, night time, rain, snow, wind, wet pavement, icy pavement, and/or an object in the road. In some examples, the cognitive ability analyzer 184 processes the image data 145 with one or more computer vision algorithms to detect driving behaviors and/or driving conditions. Additionally and/or alternatively, the cognitive ability analyzer 184 can process the image data 145 with one or more trained machine learning models to detect driving behaviors and/or driving conditions.

The cognitive ability analyzer 184 processes detected driving behaviors and/or driving conditions in conjunction with the temporally associated physiological data 155 to determine one or more biomarkers representative of cognitive decline. In some examples, the detected driving behaviors and/or driving conditions, and the physiological data 155 are processed with one or more trained machine learning models to determine the one or more biomarkers. Accordingly, the driver assessment system 100 can continuously, unobtrusively, inexpensively, and conveniently monitor a drivers' driving ability and/or cognitive decline over time, and/or can provide an objective driving quality assessment that will be very helpful for drivers and/or their caregivers when deciding whether to cease driving.

In some examples, the cognitive ability analyzer 184 includes one or more executable programs and/or portion(s) of executable programs embodied in software and/or machine-readable instructions stored on a non-transitory or tangible machine-readable storage medium for execution by one or more processors. Additionally and/or alternatively, the cognitive ability analyzer 184 can be implemented by one or more hardware circuits structured to perform the corresponding operation(s) without executing software or instructions.

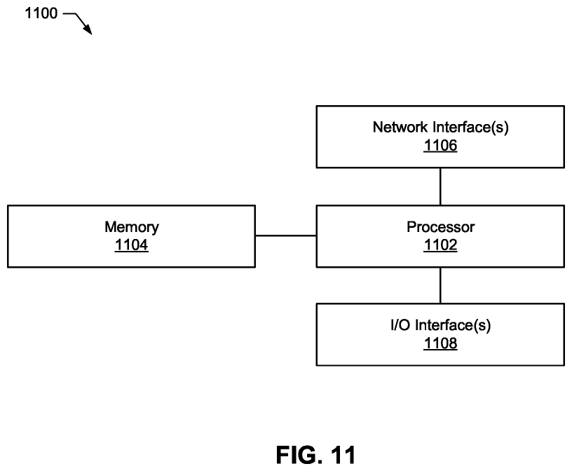

The example server 180 can be implemented by one or more physical computing devices, such as the example processing platform 1100 of FIG. 11. Additionally and/or alternatively, the server 180 can be implemented by one or more cloud-based virtual servers.

### Example Monitoring Systems

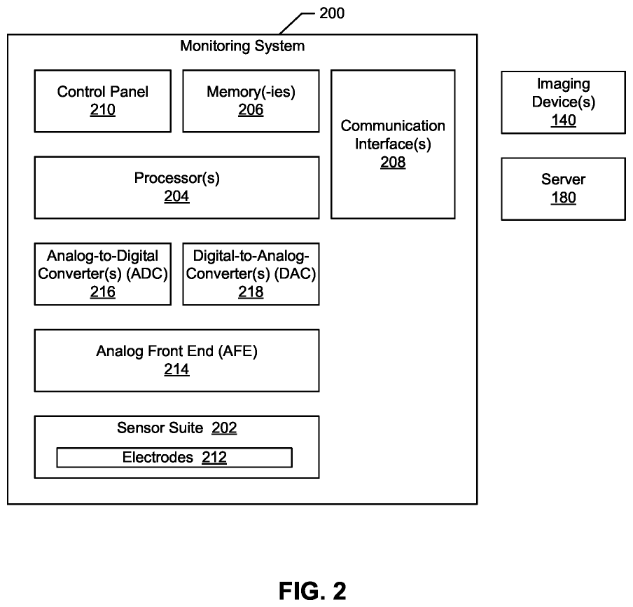

FIG. 2 is a block diagram of an example monitoring system 200 including a sensor suite 202 that can be used to implement one or more components of the example monitoring systems 110-112 of FIG. 1. The monitoring system 200 can be embedded in a steering wheel sleeve or cover 130 that is adapted to be secured to a steering wheel 170 of a vehicle 120-122. In another example, the monitoring system 200 is directly secured (e.g., affixed or adhered) to the steering wheel 170. In some examples, the monitoring system 200 is implemented on a flexible printed circuit board (PCB). The monitoring system 200 can be powered by a battery that can be recharged through a standard car charger, or other means, in some examples.

The example logic circuit of FIG. 2 is a processing platform capable of executing instructions to, for example, implement operations of the example methods described herein, as may be represented by the flowcharts of the drawings that accompany this description. Other example logic circuits capable of, for example, implementing operations of the example methods described herein include field programmable gate arrays (FPGAs) and application specific integrated circuits (ASICs).

The monitoring system 200 includes one or more processors 204 such as, for example, one or more microprocessors, controllers, and/or any suitable type of processor. The example monitoring system 200 of FIG. 2 includes non-transitory and/or tangible memory (e.g., volatile memory, non-volatile memory, etc.) 206 accessible by the processor 204 (e.g., via a memory controller). The example processor 204 interacts with the memory 206 to obtain, for example, machine-readable instructions stored in the memory 206 for, among other tasks, collecting image data 145 and physiological data 155, and conveying the image data 145 and the physiological data 155 to the server 180. Additionally or alternatively, machine-readable instructions corresponding to the example operations described herein can be stored on one or more removable media (e.g., a CD, a DVD, an SSD, removable flash memory, etc.) that can be coupled to the monitoring system 200 to provide access to the machine-readable instructions stored thereon.

The monitoring system 200 includes one or more communication interfaces 208 such as, for example, one or more network interfaces, and/or one or more input/output (I/O) interfaces for communicating with, for example, other components, devices, systems, etc. Network interface(s) enable the monitoring system 200 of FIG. 2 to communicate with, for example, another device, apparatus, system (e.g., the server 180) via, for example, one or more networks, such as the network 190. The network interface(s) can include any suitable type of network interface(s) (e.g., wired and/or wireless interfaces) configured to operate in accordance with any suitable communication protocol(s). Example network interface(s) include a TCP/IP interface, a WiFi™ transceiver (e.g., according to the IEEE 802.11x family of standards), an Ethernet transceiver, a cellular network radio, a satellite network radio, or any other suitable interface based upon any other suitable communication protocols or standards. Example I/O interface(s) include a Bluetooth® interface, a near-field communication (NFC) interface, a USB interface, a serial interface, and an infrared interface to enable receipt of user input (e.g., from a control panel 210, a mouse, a keyboard, a touch pad, a microphone, a button, etc.), and communicate output data to a user (e.g., via the control panel 210, a display, a speaker, etc.). The communication interface(s) 208 can be used to control the imaging device 140 and/or receive image data 145 from the imaging device 140, in some examples.

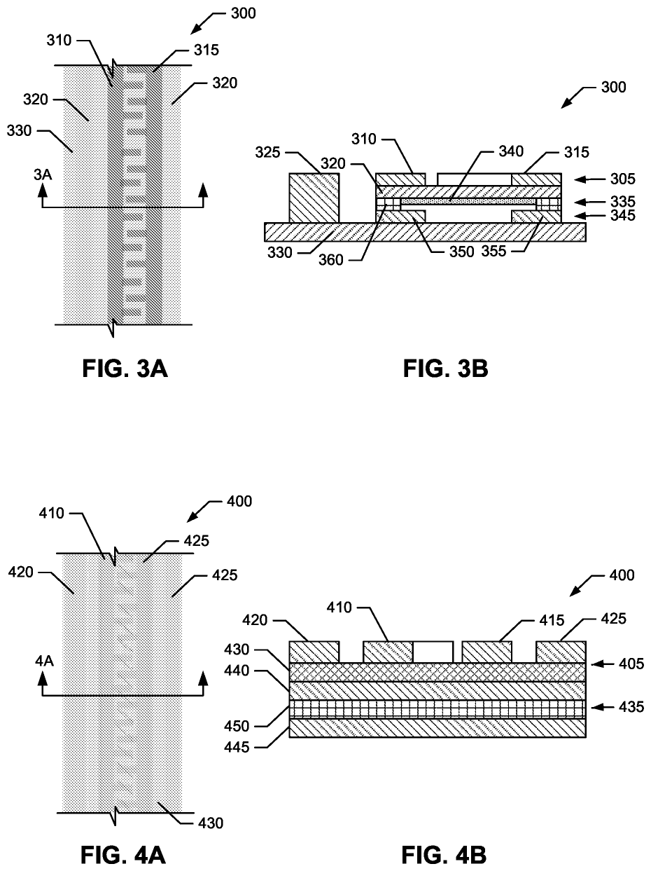

The monitoring system 200 includes the sensor suite 202 having one or more physiological sensors implemented by one or more electrodes 212 of the sensor suite 202. The electrodes 212 are configured to sense one or more physiological signals of a driver while the driver grips a steering wheel and operates a vehicle. Example physiological sensors include, but are not limited to, an EMG sensor, a heart rate or pulse sensor, a gripping force sensor, and an EDA sensor. Example implementations of the sensor suite 202 are described below in connection with FIGS. 3A, 3B, 4A, 4B, 5A, and 5B. While examples are shown in FIGS. 3A, 3B, 4A, 4B, 5A, and 5B, sensor suites having other configurations can be used.

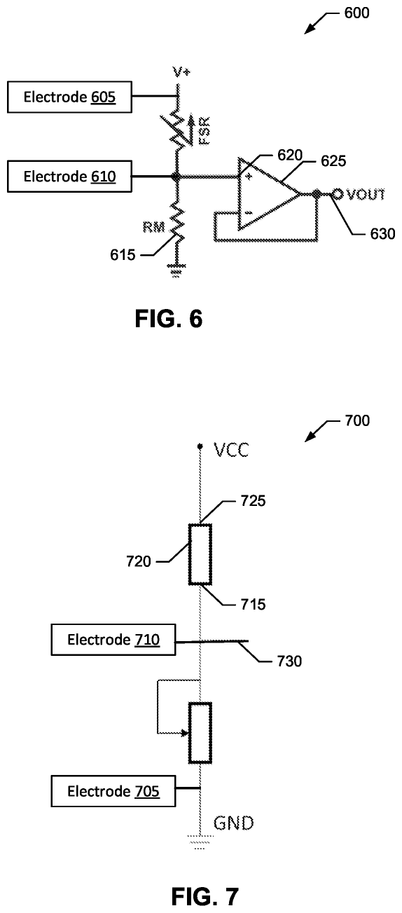

The monitoring system 200 includes an analog front end (AFE) 214 and one or more analog-to-digital converters (ADCs) 216 configured to convert physiological signals sensed by the sensor suite 202 into digital physiological data 155 that can be stored in the memory 206 and/or conveyed to the server 180 (e.g., via the communication interface(s) 208). Example circuits 600, 700 that can be used to implement portions of the AFE 214 are described below in connection with FIGS. 6 and 7. Under control of the processor 204, outputs of the AFE 214 will sampled and digitized by the ADC(s) 216.

The monitoring system 200 includes one or more digital-to-analog converters (DACs) 218 configured to convert digital control signals provided by the processor(s) 204 into one or more voltages used by the AFE 214 to sense physiological signals, for example.

### Example Sensor Suites

FIG. 3A is a top view of a portion of an example sensor suite 300 that can be used to implement (e.g., configured to operate or function as) the physiological sensors 150 of FIG. 1 and/or the sensor suite 202 of FIG. 2. FIG. 3B is cross section view of the example sensor suite 300 of FIG. 3A taken along line 3A.

The example sensor suite 300 is configured to sense hand gripping force, EMG activity, and skin impedance representative of EDA. The sensor suite 300 is comprised of multiple layers. A first or top layer 305 includes a pair of interdigitated electrodes 310 and 315 arranged on (e.g., implemented on, mounted on, or otherwise positioned on) a flexible polymer substrate 320, and a reference electrode 325 arranged on another flexible polymer substrate 330.

In the example shown, skin impedance can be measured based upon voltage and/or resistance differences between the interdigitated electrodes 310 and 315 using, for example, the example circuit 600 of FIG. 6. The electrodes 310 and 315 are interdigitated in the implementation shown to increase electrical interaction between the electrodes 310 and 315, such that the sensitivity of skin impedance measurements is increased. However, other configurations of the electrodes 310 and 315 can be used.

In the example shown, EMG activity can be measured based upon voltage and/or resistance differences measured between the reference electrode 325, and one or more of the interdigitated electrodes 310 and 315 as an active electrode. In some examples, the differences are filtered using a 10-1000 Hz bandwidth filter to eliminate 60 Hz noise and any artifacts from the environment such as vibration.

A second or middle layer 335 includes a layer 340 of a piezoresistive material whose resistance changes in response to an amount of force applied to the sensor suite 300 as a driver grips a steering wheel on which the sensor suite 300 is secured.

A third or bottom layer 345 includes another pair of interdigitated electrodes 350 and 355 that are coupled to the piezoresistive force sensing layer 340, and are on (e.g., implemented on, mounted on, or otherwise positioned on) the flexible polymer substrate 330. As shown, the interdigitated electrodes 350 and 355 can be separated from the substrate 320 with one or more spacers 360.

In the example shown, the amount of applied force applied to the sensor suite 300 can be determined by measuring the impedance between one or both of the top interdigitated electrodes 310 and 315, and one or both of the bottom interdigitated electrodes 350 and 355 using, for example, the example circuit 700 of FIG. 7.

In some examples, the electrodes 310, 315, 350 and 356 are formed of silver or a silver alloy, and the reference electrode 325 is formed of copper or a copper alloy. However, other suitable materials can be used. For example, the electrodes can be formed using screen-printing techniques using a biocompatible carbon ink. Example flexible polymer substrates are formed using parylene-C, which is a biocompatible polymer that will act as an insulating and packaging material, and provide flexibility and mechanical robustness to the sensor suite 300. Example piezoresistive materials include polyvinylidene fluoride (PVDF), doped polyaniline (PANI), and ethylene-propylene-diene-monomer (EPDM). While an example configuration and arrangement of electrodes is shown in FIGS. 3A and 3B, other suitable configurations and arrangements can be used.

The example sensor suite 300 can be formed on the flexible substrate 330 as an elongated flexible strip having a thickness of approximately 0.25 to 0.5 millimeters (mm) and a width of approximately 2.5 to 4 centimeters (cm), such that it can be embedded within a steering wheel cover or sleeve that is adapted to be installed on a steering wheel such that the sensor suite 300 extends substantially all the way around the steering wheel. In other examples, a sensor suite is formed of multiple interconnected sections of the sensor suite 300. Further still, the sensor suite 300, or multiple sections thereof, can be directed secured to a steering wheel.

FIG. 4A is a top view of a portion of another example sensor suite 400 that can be used to implement (e.g., configured to operate or function as) the physiological sensors 150 of FIG. 1 and/or the sensor suite 202 of FIG. 2. FIG. 4B is cross section view of the example sensor suite 400 of FIG. 4A taken along line 4A.

The example sensor suite 400 is configured to sense hand gripping force, EMG activity, and skin impedance representative of EDA. The sensor suite 400 is comprised of multiple layers. A first or top layer 405 includes of a pair of interdigitated electrodes 410 and 415, and a pair of EMG sensing electrodes 420 and 425 arranged on (e.g., implemented on, mounted on, or otherwise positioned on) a flexible polymer substrate 430.

In the example shown, skin impedance can be measured based upon voltage and/or resistance differences between the interdigitated electrodes 410 and 415 using, for example, the example circuit 600 of FIG. 6. The electrodes 410 and 415 are interdigitated in the implementation shown to increase electrical interaction between the electrodes 410 and 415, such that the sensitivity of skin impedance measurements is increased. However, other configurations of the electrodes 410 and 415 could be used.

In the example shown, EMG activity can be measured based upon voltage and/or resistance differences measured between one or more of the interdigitated electrodes 410 and 415 as a reference electrode, and the EMS sensing electrodes 420 and 425. In some examples, the differences are filtered using a 10-1000 Hz bandwidth filter to eliminate 60 Hz noise and any artifacts from the environment such as vibration.

A second or bottom layer 435 includes two electrode layers 440 and 445 separated by a layer 440 of a piezoresistive material whose resistance changes in response to an amount of force applied to the sensor suite 400 as a driver grips a steering wheel on which the sensor suite 400 is secured. An example piezoresistive material is piezoresistive rubber, such as velostat.

In the example shown, the amount of applied force applied to the sensor suite 400 can be determined by measuring the impedance between the electrode layers 440 and 445 using, for example, the example circuit 700 of FIG. 7.

In some examples, the electrodes 410, 415, 420, 425, 440 and 445 are formed of copper or a copper alloy. However, other suitable materials can be used. For example, the electrodes can be formed using screen-printing techniques using a biocompatible carbon ink. Example flexible polymer substrates are formed using Parylene-C. Other example piezoresistive materials include PVDF, PANI, and EPDM. While an example configuration and arrangement of electrodes is shown in FIGS. 4A and 4B, other suitable configurations and arrangements can be used.

The example sensor suite 400 can be formed on a flexible substrate as an elongated flexible strip having a thickness of approximately 0.5 to 1 mm and a width of approximately 2.5 to 4 cm, such that it can be embedded within a steering wheel cover or sleeve that is adapted to be installed on a steering wheel such that the sensor suite 400 extends substantially all the way around the steering wheel. In other examples, a sensor suite is formed of multiple interconnected sections of the sensor suite 400. Further still, the sensor suite 400, or multiple sections thereof, can be directly secured to a steering wheel.

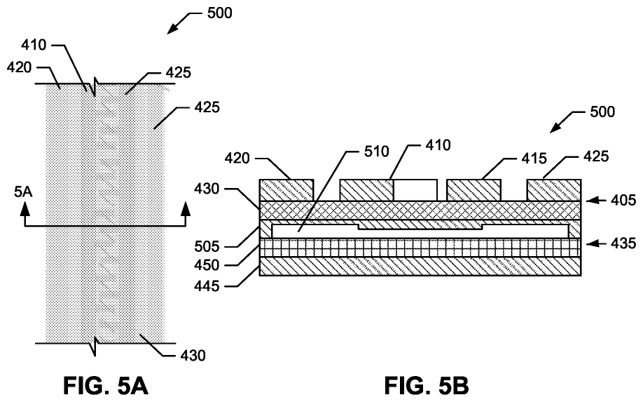

FIG. 5A is a top view of a portion of yet another example sensor suite 500 that can be used to implement (e.g., configured to operate or function as) the physiological sensors 150 of FIG. 1 and/or the sensor suite 202 of FIG. 2. FIG. 5B is cross section view of the example sensor suite 500 of FIG. 5A taken along line 5A. The example sensor suite 500 is similar to the sensor suite 400 of FIGS. 4A and 4B. Like elements are shown with like reference numbers in FIGS. 4A, 4B, 5A and 5B, and descriptions of the like elements are not repeated here. Instead, the interested reader is referred to the descriptions of like elements provided above in connection with FIGS. 4A and 4B.

Compared to FIGS. 4A and 4B, the electrode layer 440 is replaced with a differently shaped electrode layer 505. As shown, the cross section of the electrode layer 505 is shaped to form at least a partial air gap 510 between the electrode layer 505 and the layer 440 of piezoresistive material. The introduction of the air gap 510 can improve the stability and/or reliability of force sensing. A similar air gap is also shown in FIG. 3B. However, as shown in FIG. 4B, the air gap 510 can be eliminated.

The example sensor suite 500 can be formed on (e.g., implemented on, mounted on, or otherwise positioned on) an elongated substrate as an elongated flexible strip having a thickness of approximately 0.5 to 1 mm and an overall width of approximately 2.5 to 4 cm, such that it can be embedded within a steering wheel cover or sleeve that is adapted to be installed on a steering wheel such that the sensor suite 500 extends substantially all the way around the steering wheel. In other examples, a sensor suite is formed of multiple interconnected sections of the sensor suite 500. Further still the sensor suite 500, or multiple sections thereof can be directed secured to a steering wheel.

In some examples, electrodes of the sensor suite 500 are approximately 0.3 to 30 micrometers (m) thick. In some examples, the fingers of the interdigitated electrodes 410 and 415 are approximately 0.5 to 1 mm in length, and are separated from the other electrode 410, 415 by approximately 0.5 to 1 mm. However, other dimensions can be used.

### Example Analog Front End (AFE) Circuits

FIG. 6 is a schematic diagram of example analog circuit 600 that can be used to, for example, measure a skin impedance representing EDA. The analog circuit 600 can be used to implement a portion of the AFE 214 of FIG. 2. The example circuit 600 is arranged in an example voltage divider configuration, wherein a supply voltage V+ is applied to a first electrode 605 of a pair of interdigitated electrodes (e.g., the electrodes 310, 315, or the electrodes 410, 415), and the second electrode 610 of the pair of electrodes is connected to a measuring resistor 615 and the positive input terminal 620 of an amplifier 625 in a voltage divider configuration, in the implementation shown. An output voltage Vout 630 of the amplifier 625 can be converted to a digital value representing the voltage Vout 630 by an ADC 216, and provided to the processor 204 for storage and/or communication to the server 180. The output voltage Vout 630 of the amplifier 625 can be expressed mathematically as:

$$\\ \begin{matrix}V out = \frac{{R}_{M} {V}^{+}}{{R}_{M} + {R}_{FSR}} , & EQN ( 1 )\end{matrix}$$

where R_{M} is the resistance of the measuring resistor 615, and R_{FSR} is the resistance of a force sensing layer (e.g., the layer 340, 450, or 505), which varies as an applied force changes. Knowing the measured voltage Vout 630, or a digital representation of the measured voltage Vout, V+, and Rₘ, the processor 204 can solve for R_{FSR} using EQN (1). In some examples, the relationship between R_{FSR} and force is not be linear (e.g., parabolic). In such examples, R_{FSR} can be converted to force using, for example, a piece-wise linear curve that approximates the non-linear relationship between R_{FSR} and force. An example supply voltage V+ is five (5) volts direct current (DC), and an example measuring resistor 615 has a resistance of 3.3 kΩ. In some examples, a DAC 218 can be used by the processor 204 to provide the supply voltage V+, however, it can instead be a supply voltage already being provided for a measuring system, for example.

FIG. 7 is a schematic diagram of example analog circuit 700 that can be used to, for example, measure a gripping force. The analog circuit 700 can be used to implement a portion of the AFE 214 of FIG. 2. The example circuit 700 is arranged in another example voltage divider configuration, wherein a first electrode 705 of a pair of interdigitated electrodes (e.g., the electrodes 310, 315, or the electrodes 410, 415) is connected to ground GND, and the second electrode 710 of the pair of electrodes is connected to one terminal 715 of a reference resistor 720, where the other terminal 725 of the reference resistor 720 is connected to a supply voltage Vcc. A voltage 730 measured at the second electrode 710 can be converted to a digital value representing the voltage 730 by an ADC 216, and provided to the processor 204 for storage and/or communication to the server 180. The voltage 730 varies linearly with a ratio of the impedance of the resistor 720 to the impedance of the skin at the pair of interdigitated electrodes, and can be solved by the processor 204 to determine skin impedance. In some examples, the supply voltage Vcc is a DC voltage. However, the supply voltage Vcc can be provided at different frequencies such that skin impedance can be measured at different frequencies. The processor 204 can generate such a supply voltage by, for example, generating a sequence of digital values representing a sine wave, and converting them to an analog voltage using a DAC 218.

### Example Cognitive Ability Analyzers

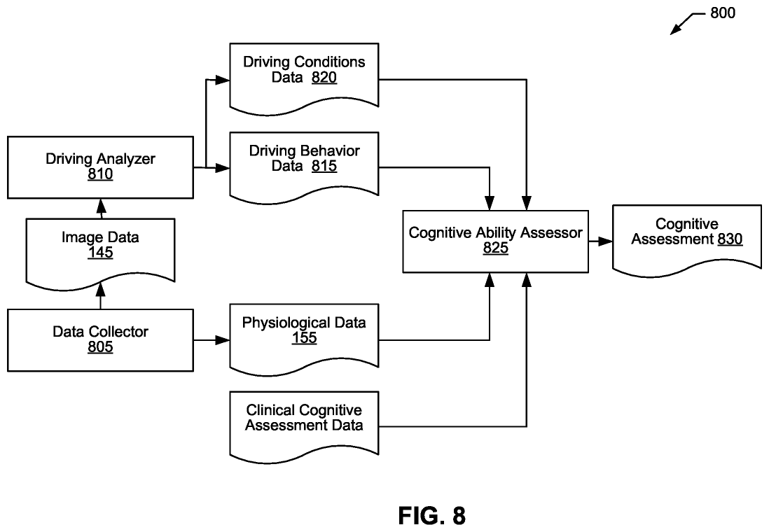

FIG. 8 is a block diagram of an example cognitive ability analyzer 800 that can be used to implement (e.g., configured to operate or function as) the example cognitive ability analyzer 184 of FIG. 1. The example cognitive ability analyzer 800 includes a data collector 805 configured to collect, receive, or otherwise obtain image data 145 and physiological data 155 from monitoring systems 110-112, 200, and store the data in one or more tangible or non-transitory storage devices, such as the device(s) 182.

The cognitive ability analyzer 800 includes a driving analyzer 810 configured to process the image data 145 to determine driving behavior data 815 representing driving behaviors of a driver. Example driving behaviors include an ability to stay within an intended lane (e.g., lane deviations), maintain inter-vehicle distances, demonstrate appropriate reaction time when exposed to an unexpected hazard or driving condition (such as an object in the road or a stopped vehicle), maintain a speed that fits within the expected range of current traffic (e.g., not too fast, but not too slow), recognize and react to stop signs, and/or any other driving behaviors. In some examples, the driving analyzer 810 can also process the image data 145 to detect driving conditions data 820 representing conditions such as daytime, nighttime, rain, snow, window, wet pavement, icy pavement, and an object in the road. In some examples, the driving analyzer 810 processes the image data 145 with any number and/or type(s) of computer vision algorithms to detect driving behaviors and/or driving conditions. Additionally and/or alternatively, the driving analyzer 810 can process the image data 145 with one or more trained machine learning models to detect driving behaviors and/or driving conditions. In some examples, such machine learning models can be trained using, for example, supervised learning. For example, machine learning model(s) being trained can process incoming image data 145 collected for a large number of drivers over time to identify respective driving behaviors and/or driving conditions. The driving behaviors and/or driving conditions identified by the machine learning model(s) can be compared to driving behaviors and/or driving conditions determined using other techniques, such as computer vision and/or human manual classification. Differences can then be used to update the machine learning models.

The cognitive ability analyzer 800 includes a cognitive ability assessor 825 that processes the driving behaviors data 815 and/or the driving conditions data 820 in conjunction with temporally associated physiological data 155 to determine a cognitive assessment 830 that includes one or more biomarkers representative of cognitive decline. In some examples, the cognitive ability assessor 825 processes the data 155, 815, and 820 with one or more trained machine learning models to determine the cognitive assessment. In some examples, such machine learning models can be trained using, for example, supervised learning. For example, the machine learning model(s) being trained can process driving behaviors data 815, driving conditions data 820, and physiological data 155 collected for a large number of drivers over time to determine respective cognitive assessments. For example, data can be collected for drivers with varying levels of known cognitive decline. Those cognitive assessments can be compared with cognitive assessments made using other techniques, such as clinical assessment. Differences can then be used to update the machine learning model(s).

In some examples, the cognitive ability assessor 825 also processes clinical cognitive assessment data 835, when available. Example clinical cognitive assessment data 835 includes results of the MMSE, or any other objective and/or subjective clinical assessment.

### Example Machine Learning Frameworks

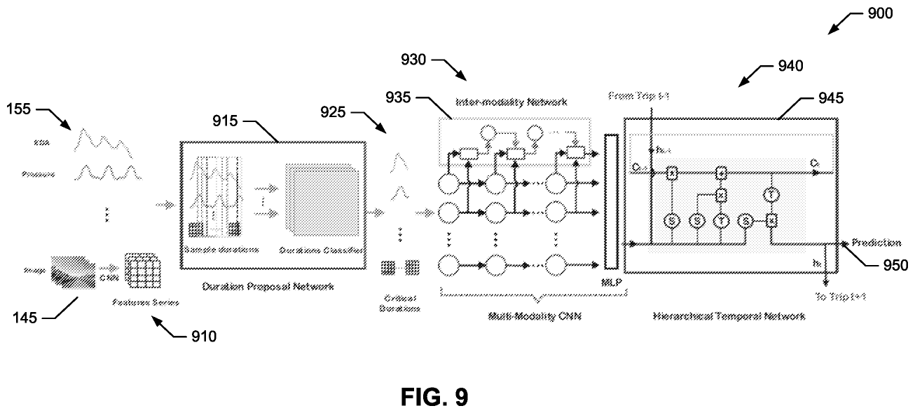

FIG. 9 is a block diagram of an example machine learning framework, model, or architecture 900 that can be configured to implement (e.g., configured to operate or function as) the driving analyzer 810, the cognitive ability assessor 825, and/or, more generally, the cognitive ability analyzers 184 and 800. The example machine learning framework 900 utilizes deep-learning based analytics for monitoring and prediction of cognitive status across multiple drivers over time.

The machine learning framework 900 includes one or more convolutional neural networks (CNNs) 910 trained and configured to classify various features of interest (e.g., driving behaviors 815 and/or driving conditions 820 of interest) from collected image data 145.

The machine learning framework 900 includes one or more trained duration proposal networks 915 trained and configured to identify periods, portions, segment, intervals, or durations of interest in the image data 145 and/or the physiological data 155. Cognitively impaired drivers, and young and/or healthy drivers are expected to share similar driving behaviors and physiological states during situations that do not involve complex cognitive activities, e.g., straight driving with light traffic. Accordingly, the duration proposal network(s) 915 are trained and configured to identify such common driving situations such that the data 145, 155 collected during those situations are not used to assess driving ability and/or cognitive decline. The duration proposal network(s) 915 are further trained and configured to identify critical situations that can best represent the cognitive impairment level of the drivers over time. In some examples, the duration proposal network(s) 915 include one or more CNNs trained to learn the situations of interests from a constructed set of sub-intervals for different situations using similarity functions of the sub-intervals as loss functions. The duration proposal network(s) 915 are trained and configured to only retain the data 925 associated with periods, portions, segment, intervals, or durations that are expected to improve learning efficiency and/or the classification performance. Thus, the data 925 represents a subset of the data 145, 155.

The machine learning framework 900 includes one or more cross-modality CNNs 930 trained and configured to recognize intra-modality and/or cross-modality correlations. For example, to recognize cross-modality correlations between physiological data and driving behaviors. For example, a driver keeps getting nervous (e.g., as reflected in EDA or HR signals) when driving on a congested road segment (e.g., as reflected in the number of vehicles detected in associated image data). In some examples, the cross-modality CNN(s) 930 have a CNN for each channel or modality, and fuses features identified by the CNNs at multiple stages (e.g., see box 935) to identify cross-modality correlations. For example, parameters of the cross-modality CNNs and the channel-specific CNNs can be jointly trained using a global loss function that combines the regression/classification errors from both networks.

The machine learning framework 900 includes one or more temporal networks 940 trained and configured to monitor the progression of cognitive impairment in drivers based upon the data from the most recent trip and information aggregated from past trips. In some examples, the temporal network(s) 940 implement (e.g., configured to operate or function as) a long short term memory (LSTM) network 945 to aggregate information from past trips. Example inputs for the LSTM network 945 are extracted features from the multi-modality CNN(s) 930, and its outputs 950 can be fed into a multi-layer perception (MLP) network (not shown for clarity of illustration) to predict a cognitive impairment level. The example hierarchical temporal network(s) 940 not only utilize the time-series data for each trip, but also connect the trips to capture the temporal dependence across multiple trips.

### Example Flowchart

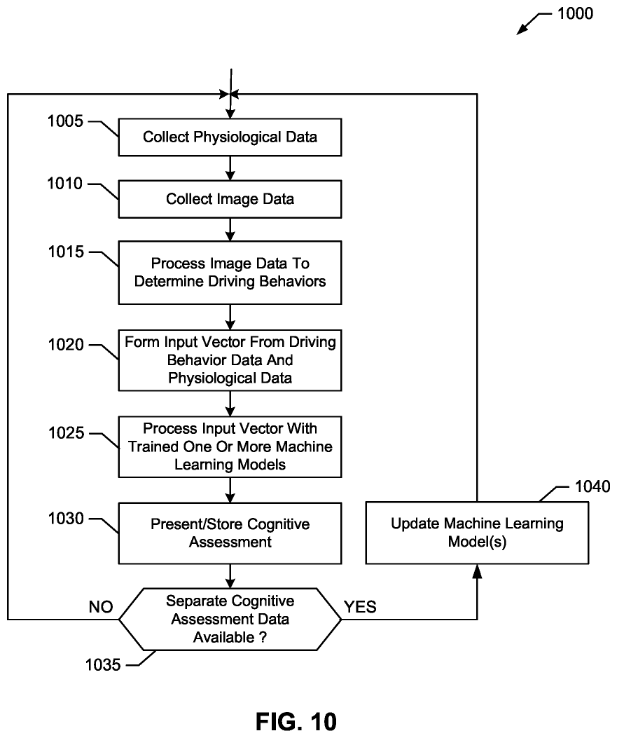

FIG. 10 is a flowchart 1000 representative of an example method, hardware logic, machine-readable instructions, or software for assessing cognitive decline based upon monitored driving performance, as disclosed herein. Any or all of the blocks of FIG. 10 can be an executable program or portion(s) of an executable program embodied in software and/or machine-readable instructions stored on a non-transitory, machine-readable storage medium for execution by one or more processors such as the processor 1102 of FIG. 11. Additionally and/or alternatively, any or all of the blocks of FIG. 10 can be implemented by one or more hardware circuits structured to perform the corresponding operation(s) without executing software or instructions.

The example flowchart 1000 begins at block 1005 with, for example, the cognitive ability analyzer 184, 800 collecting physiological data 155 (block 1005), and associated image data 145 (block 1010). The cognitive ability analyzer 184, 800 processes the image data to determine driving behaviors and/or driving conditions (block 1015). The cognitive ability analyzer 184, 800 forms an input vector including at least a portion of the image data and the driving behaviors data 815 and/or the driving conditions data 820 (block 1020), and processes the input vector with one or more trained machine learning models (block 1025) to make a driving and/or cognitive assessment. As appropriate, the driving and/or cognitive assessment is presented and/or stored (block 1030). If additional cognitive assessment data is available (e.g., clinical assessment data) (block 1035), one or more differences between the assessment made by the machine learning model(s) and the additional assessment data can be used to update the machine learning model(s) (block 1040), and control returns to block 1005 to collect next data. Otherwise (block 1035), control simply returns to block 1005 to collect more data.

### Example Processing Platform

FIG. 11 is a block diagram representative of an example logic circuit capable of implementing, for example, one or more components of the example server 180. The example logic circuit of FIG. 11 is a processing platform 1100 capable of executing instructions to, for example, implement operations of the example methods described herein, as may be represented by the flowcharts of the drawings that accompany this description. Other example logic circuits capable of, for example, implementing operations of the example methods described herein include FPGAs and ASICs.

The example processing platform 1100 of FIG. 11 includes a processor 1102 such as, for example, one or more microprocessors, controllers, and/or any suitable type of processor. The example processing platform 1100 of FIG. 11 includes memory (e.g., volatile memory, non-volatile memory) 1104 accessible by the processor 1102 (e.g., via a memory controller). The example processor 1102 interacts with the memory 1104 to obtain, for example, machine-readable instructions stored in the memory 1104 corresponding to, for example, the operations represented by the flowcharts of this disclosure. Additionally or alternatively, machine-readable instructions corresponding to the example operations described herein can be stored on one or more removable media (e.g., a CD, a DVD, an SSD, removable flash memory, etc.) that can be coupled to the processing platform 1100 to provide access to the machine-readable instructions stored thereon.

The example processing platform 1100 of FIG. 11 includes one or more communication interfaces such as, for example, one or more network interfaces 1106, and/or one or more I/O interfaces 1108. The communication interface(s) enable the processing platform 1100 of FIG. 11 to communicate with, for example, another device, apparatus, system (e.g., the monitoring systems 110-112 and 200), datastore, database, and/or any other machine. The communication interface(s) 208 can be used to control the imaging device 140 and/or receive image data 145 from the imaging device 140.

The example processing platform 1100 of FIG. 11 includes the network interface(s) 1106 to enable communication with other machines (e.g., the monitoring systems 110-112 and 200) via, for example, one or more networks such as the network 190. The example network interface 1106 includes any suitable type of communication interface(s) (e.g., wired and/or wireless interfaces) configured to operate in accordance with any suitable communication protocol(s). Example network interfaces 1106 include a TCP/IP interface, a WiFi™ transceiver (e.g., according to the IEEE 802.11x family of standards), an Ethernet transceiver, a cellular network radio, a satellite network radio, or any other suitable interface based upon any other suitable communication protocols or standards.

The example, processing platform 1100 of FIG. 11 includes the input/output (I/O) interface(s) 1108 (e.g., a Bluetooth® interface, an NFC interface, a USB interface, a serial interface, an infrared interface, etc.) to enable receipt of user input (e.g., a touch screen, keyboard, mouse, touch pad, joystick, trackball, microphone, button, etc.) and communication of output data (e.g., driving and/or cognitive assessments, instructions, data, images, etc.) to the user (e.g., via a display, speaker, printer, etc.).

The above description refers to block diagrams of the accompanying drawings. Alternative implementations of the examples represented by the block diagrams include one or more additional or alternative elements, processes and/or devices. Additionally and/or alternatively, one or more of the example blocks of the diagrams can be combined, divided, re-arranged, and/or omitted. Components represented by blocks of the diagrams can be implemented by hardware, software, firmware, and/or any combination of hardware, software, and/or firmware. In some examples, at least one of the components represented by the blocks is implemented by a logic circuit. As used herein, the term “logic circuit” is expressly defined as a physical device including at least one hardware component configured (e.g., via operation in accordance with a predetermined configuration and/or via execution of stored machine-readable instructions) to control one or more machines and/or perform operations of one or more machines. Examples of a logic circuit include one or more processors, one or more coprocessors, one or more microprocessors, one or more controllers, one or more digital signal processors (DSPs), one or more ASICs, one or more FPGAs, one or more microcontroller units (MCUs), one or more hardware accelerators, one or more special-purpose computer chips, and one or more system-on-a-chip (SoC) devices. Some example logic circuits, such as ASICs or FPGAs, are specifically configured hardware for performing operations (e.g., one or more of the operations described herein and represented by the flowcharts of this disclosure, if such are present). Some example logic circuits are hardware that executes machine-readable instructions to perform operations (e.g., one or more of the operations described herein and represented by the flowcharts of this disclosure, if such are present). Some example logic circuits include a combination of specifically configured hardware and hardware that executes machine-readable instructions. The above description refers to various operations described herein and flowcharts that may be appended hereto to illustrate the flow of those operations. Any such flowcharts are representative of example methods disclosed herein. In some examples, the methods represented by the flowcharts implement the system represented by the block diagrams. Alternative implementations of example methods disclosed herein may include additional or alternative operations. Further, operations of alternative implementations of the methods disclosed herein may combined, divided, re-arranged, and/or omitted. In some examples, the operations described herein are implemented by machine-readable instructions (e.g., software and/or firmware) stored on a medium (e.g., a tangible machine-readable medium) for execution by one or more logic circuits (e.g., processor(s)). In some examples, the operations described herein are implemented by one or more configurations of one or more specifically designed logic circuits (e.g., ASIC(s)). In some examples the operations described herein are implemented by a combination of specifically designed logic circuit(s) and machine-readable instructions stored on a medium (e.g., a tangible machine-readable medium) for execution by logic circuit(s).

Example systems, apparatus, and methods for assessing cognitive decline based upon monitored driving performance are disclosed herein. Further examples and combinations thereof include at least the following.

Example 1 is a system for assessing cognitive decline, the system comprising:
a sensor suite configured to be used in conjunction with a vehicle, the sensor suite comprising:
   one or more sensors configured to sense one or more physiological signals of a driver while the driver grips the steering wheel and operates the vehicle,
   one or more converters configured to convert the one or more sensed physiological signals into physiological data representative of one or more physiological states of the driver while they operate the vehicle, and
   an imaging device configured to capture image data representative of the vehicle within an environment of use; and
a computing device comprising one or more processors configured to:
   receive the physiological data and the image data from the sensor suite, and
   determine one or more biomarkers representative of cognitive decline based upon the physiological data and the image data.

Example 2 is the system of example 1, wherein the one or more sensors comprise at least one of an electrodermal activity sensor, a heart rate sensor, an electromyography sensor, or a gripping force sensor.

Example 3 is the system of example 1:
wherein the sensor suite comprises a flexible elongated strip comprising at least a first layer and a second layer;
wherein the first layer comprises a pair of interdigitated electrodes configured to implement an electrodermal activity sensor; and
wherein the second layer is configured to implement a force sensor, the second layer comprising:
   a first electrode layer,
   a second electrode layer, and
   a force-sensitive layer between the first electrode layer and the second electrode layer, the force-sensitive layer having an electrical resistance that varies with a force applied to the force sensing layer by the first and second electrode layers in response to a force exerted on the steering wheel by one or more of the driver's hands.

Example 4 is the system of example 3, wherein the first layer further comprises a reference electrode configured to, in conjunction with at least one of the pair of interdigitated electrodes, to implement an electromyography sensor.

Example 5 is the system of example 3, wherein the force sensing layer comprises a piezoresistive material.

Example 6 is the system of example 3, wherein at least one of the first and second electrode layers is at least partially separated from the force sensing layer by an air gap.

Example 7 is the system of example 1, wherein the one or more sensors are implemented on one or more elongated flexible substrates adapted to be secured to the steering wheel.

Example 8 is the system of example 7, wherein the one or more elongated flexible substrates are integrated into a steering wheel sleeve or cover adapted to be installed on the steering wheel to secure the one or more elongated flexible substrate to the steering wheel.

Example 9 is the system of example 7, wherein the one or more elongated flexible substrates are adapted to be directly affixed to the steering wheel.

Example 10 is the system of any one of example 1 to example 9, wherein the sensor suite is configured to:
sense one or more physiological signals of the driver while the driver grips the steering wheel to operate the vehicle;
convert the one or more sensed physiological signals into physiological data representative of one or more physiological states of the driver while they operate the vehicle;
capture image data representative of the vehicle within an environment of use; and
communicate the physiological data and the image data to a computing device.

Example 11 is the system of any one of example 1 to example 9, wherein the one or more processors are configured to determine the biomarkers by:
determining, based upon the image data, one or more driving behaviors of the driver;
determining, based upon the physiological data, one or more physiological states of the driver that temporally overlap the one or more driving behaviors; and
determining the one or more biomarkers based upon the driving behaviors and the physiological states.

Example 12 is the system of example 11, wherein the one or more processors are further configured to:
determine, based upon the image data, one or more driving conditions; and
determine the one or more biomarkers based upon the driving behaviors, the driving conditions, and the physiological states.

Example 13 is the system of example 11, wherein the one or more driving behaviors include at least one of a driving lane deviation, an inappropriate inter-vehicle distance, or a missed stop sign event.

Example 14 is the system of example 11, wherein the one or more driving behaviors are determined using one or more computer vision algorithms.

Example 15 is the system of example 11, wherein the one or more driving behaviors are determined using one or more trained machine learning models.

Example 16 is the system any one of example 1 to example 9, wherein the one or more processors are configured to determine the biomarkers by:
forming one or more input vectors representing the physiological data and the image data; and
processing, with one or more trained machine learning models, the one or more input vectors to determine the one or more biomarkers.

Example 17 is the system of example 16, wherein the computing device is remote from the vehicle, and wherein the sensor suite further comprises a communication interface configured to convey the physiological data and the image data to the computing device.

Example 18 is the system of any one of example 1 to example 9, wherein the computing device is configured to:
receive physiological data representing one or more physiological states of a driver while they operate a vehicle, the physiological data captured using one or more sensors secured to a steering wheel used by the driver to operate the vehicle;
receive imaging device representative of the vehicle within an environment of use; and
determine one or more biomarkers representative of cognitive decline based upon the physiological data and the image data.

Example 19 is an apparatus, comprising:
one or more sensors adapted to be secured to a steering wheel of a vehicle, and configured to sense one or more physiological signals of a driver while the driver grips the steering wheel and operates the vehicle;
one or more converters configured to convert the one or more sensed physiological signals into physiological data representative of one or more physiological states of the driver while they operate the vehicle;
an imaging device configured to capture image data representative of the vehicle within an environment of use; and
a communication interface to transfer the physiological data and the image data to a computing device.

Example 20 is the apparatus of example 19, wherein the one or more sensors are implemented on one or more elongated flexible substrates adapted to be secured to the steering wheel.

Example 21 is the apparatus of example 20, wherein the one or more substrates are integrated into a steering wheel sleeve adapted to be installed on the steering wheel to secure the one or more sensors to the steering wheel.

Example 22 is the apparatus of example 20, wherein the one or more elongated flexible substrates are adapted to be directly affixed to the steering wheel.

Example 23 is the apparatus of any one of example 19 to example 22, wherein the one or more sensors comprise at least one of an electrodermal activity sensor, a heart rate sensor, an electromyography sensor, or a gripping force sensor.

Example 24 is the apparatus of any one of example 19 to example 22, further comprising an elongated strip comprising at least a first layer and a second layer,
wherein the first layer comprises a pair of interdigitated electrodes configured to implement an electrodermal activity sensor, and
wherein the second layer is configured to implement a force sensor, and comprises:
   a first electrode layer,
   a second electrode layer, and
   a force-sensitive layer between the first electrode layer and the second electrode layer, the force-sensitive layer having an electrical resistance that varies with a force applied to the force sensing layer by the first and second electrode layers in response to a force exerted on the steering wheel by one or more of the driver's hands.

Example 25 is the apparatus of example 24, wherein the first layer further comprises a reference electrode configured to, in conjunction with at least one of the pair of interdigitated electrodes, to implement an electromyography sensor.

Example 26 is the apparatus of example 24, wherein the force sensing layer comprises a piezoresistive material.

Example 27 is the apparatus of example 24, wherein at least one of the first and second electrode layers is at least partially separated from the force sensing layer by an air gap.

Example 28 is a method, comprising:
sensing, with one or more sensors of a sensor suite adapted to be secured to a steering wheel of a vehicle, one or more physiological signals of a driver while the driver grips the steering wheel to operate the vehicle;
converting the one or more sensed physiological signals into physiological data representative of one or more physiological states of the driver while they operate the vehicle;
capturing image data representative of the vehicle within an environment of use; and
communicating the physiological data and the image data to a computing device.

Example 29 is the method of example 28, wherein the one or more sensors comprise at least one of an electrodermal activity sensor, a heart rate sensor, an electromyography sensor, or a gripping force sensor.

Example 30 is the method of either one of example 28 and example 29, further comprising:
sensing, with a pair of interdigitated electrodes on a first layer of the sensor suite, an electrodermal activity physiological signal; and
sensing, with a first electrode layer, a force-sensitive layer, and a second electrode layer on a second layer of the sensor suite, a gripping force exerted on the steering wheel by one or more of the driver's hands.

Example 31 is the method of example 30, further comprising:
sensing, with a reference electrode on the first layer and one or both of the pair of interdigitated electrodes, an electromyography signal.

Example 32 is a method, comprising:
receiving physiological data representing one or more physiological states of a driver while they operate a vehicle, the physiological data captured using one or more sensors secured to a steering wheel used by the driver to operate the vehicle;
receiving imaging device representative of the vehicle within an environment of use; and
determining one or more biomarkers representative of cognitive decline based upon the physiological data and the image data.

Example 33 is the method of example 32, wherein determining the one or more biomarkers comprises:
determining, based upon the image data, one or more driving behaviors of the driver;
determining, based upon the physiological data, one or more physiological states of the driver that temporally overlap the one or more driving behaviors; and
determining the one or more biomarkers based upon the driving behaviors and the physiological states.

Example 34 is the method of example 33, further comprising:
determining, based upon the image data, one or more driving conditions; and
determining the one or more biomarkers based upon the driving behaviors, the driving conditions, and the physiological states.

Example 35 is the method of example 33, wherein the one or more driving behaviors include at least one of a driving lane deviation, an inter-vehicle distance, or a missed stop sign event.

Example 36 is the method of any one of example 33 to example 35, further comprising:
determining the one or more driving behaviors using one or more computer vision algorithms.

Example 37 is the method of any one of example 33 to example 35, further comprising:
determining the one or more driving behaviors using one or more trained machine learning models.

Example 38 is the method of example 32, wherein determining the biomarkers comprises:
forming one or more input vectors representing the physiological data and the image data; and
processing, with one or more trained machine learning models, the one or more input vectors to determine the one or more biomarkers.

Example 39 is a tangible machine-readable storage medium storing instructions that, when executed by one or more processors, cause a machine to:
receive physiological data representing one or more physiological states of a driver while they operate a vehicle, the physiological data captured using one or more sensors secured to a steering wheel used by the driver to operate the vehicle;
receive imaging device representative of the vehicle within an environment of use; and
determine one or more biomarkers representative of cognitive decline based upon the physiological data and the image data.

Example 40 is the storage medium of example 39, wherein the instructions, when executed by one or more processors, cause the machine to determine the one or more biomarkers by:
determining, based upon the image data, one or more driving behaviors of the driver;
determining, based upon the physiological data, one or more physiological states of the driver that temporally overlap the one or more driving behaviors; and
determining the one or more biomarkers based upon the driving behaviors and the physiological states.

Example 41 is the storage medium of example 40, wherein the instructions, when executed by one or more processors, cause the machine to:
determine, based upon the image data, one or more driving conditions; and
determine the one or more biomarkers based upon the driving behaviors, the driving conditions, and the physiological states.

Example 42 is the storage medium of example 40, wherein the one or more driving behaviors include at least one of a driving lane deviation, an inappropriate inter-vehicle distance, or a missed stop sign event.

Example 43 is the storage medium of any one of example 40 to example 42, wherein the instructions, when executed by one or more processors, cause the machine to:
determine the one or more driving behaviors using one or more computer vision algorithms.

Example 44 is the storage medium of any one of example 40 to example 42, wherein the instructions, when executed by one or more processors, cause the machine to:
determine the one or more driving behaviors using one or more trained machine learning models.

Example 45 is the storage medium of example 39, wherein the instructions, when executed by one or more processors, cause the machine to determine the one or more biomarkers by:
forming one or more input vectors representing the physiological data and the image data; and
processing, with one or more trained machine learning models, the one or more input vectors to determine the one or more biomarkers.

Example 46 is a system for monitoring driving performance, the system comprising:
(a) a physiological sensor suite adapted to sense (i) bio-electrical signals and (ii) pressure signals when in contact with human skin (e.g., a hand or hands);
(b) a driving camera;
(c) a computer system adapted to (i) receive data from the physiological sensor suite and the driving camera and (ii) evaluate stress/cognitive biomarkers and computer vision algorithms based on the same; and
(d) (optionally) a wireless communication network module adapted to transfer data received by the computer system to a remote network for collective data processing.

Example 47 is the system of example 46, wherein the physiological sensor suite is flexible and adapted to be mounted on a steering wheel of a vehicle.

The system of claim 1, wherein the physiological sensor suite has a layered structure comprising a top electrode layer, a bottom electrode layer, and an intermediate piezoresistive layer between the top electrode layer and the bottom electrode layer.

Example 48 is the system of example 46, wherein the physiological sensor suite is adapted to sense and measure physiological signals from a user selected from the group consisting of electrodermal activity (EDA), heart rate (HR), electromyography (EMG), hand pressure (or force), and combinations thereof.

Example 49 is the system of example 46, wherein, based on received data from the driving camera, the computer system is adapted to detect and determine one or more driving states selected from the group consisting of driving lane deviation, inter-vehicle distance, missed STOP events, and combinations thereof.

Example 50 is a vehicle comprising the system of example 46, wherein:
the physiological sensor suite is mounted to a steering wheel of the vehicle;
the driving camera is mounted to the vehicle in a position that permits viewing of the environmental surroundings while driving the vehicle; and
the computer system is mounted in the vehicle and is communication-coupled to the physiological sensor suite and the driving camera.

### Additional Considerations

Because other modifications and changes varied to fit particular operating requirements and environments will be apparent to those skilled in the art, the disclosure is not considered limited to the example chosen for purposes of illustration and covers all changes and modifications which do not constitute departures from the true spirit and scope of this disclosure.

Accordingly, the foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In case of conflict, the present description, including definitions, will control.

As used herein, each of the terms “tangible machine-readable medium,” “non-transitory machine-readable medium” and “machine-readable storage device” is expressly defined as a storage medium (e.g., a platter of a hard disk drive, a digital versatile disc, a compact disc, flash memory, read-only memory, random-access memory, etc.) on which machine-readable instructions (e.g., program code in the form of, for example, software and/or firmware) are stored for any suitable duration of time (e.g., permanently, for an extended period of time (e.g., while a program associated with the machine-readable instructions is executing), and/or a short period of time (e.g., while the machine-readable instructions are cached and/or during a buffering process)). Further, as used herein, each of the terms “tangible machine-readable medium,” “non-transitory machine-readable medium” and “machine-readable storage device” is expressly defined to exclude propagating signals. That is, as used in any claim of this patent, none of the terms “tangible machine-readable medium,” “non-transitory machine-readable medium,” and “machine-readable storage device” can be read to be implemented by a propagating signal.

In the foregoing specification, specific examples have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described examples/examples/implementations should not be interpreted as mutually exclusive, and should instead be understood as potentially combinable if such combinations are permissive in any way. In other words, any feature disclosed in any of the aforementioned examples/examples/implementations may be included in any of the other aforementioned examples/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or system that comprises, has, includes, contains a list of elements does not include only those elements but may include other elements not expressly listed or inherent to such process, method, article, or system. An element proceeded by “comprises . . . a”, “has . . . a”, “includes . . . a”, “contains . . . a” does not, without more constraints, preclude the existence of additional identical elements in the process, method, article, or system that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting example the term is defined to be within 10%, in another example within 5%, in another example within 1% and in another example within 0.5%. The term “coupled” as used herein is defined as connected, although not necessarily directly and not necessarily mechanically. A device or structure that is “configured” in a certain way is configured in at least that way, but may also be configured in ways that are not listed.

Further, unless expressly stated to the contrary, “or” refers to an inclusive or and not to an exclusive or. For example, “A, B or C” refers to any combination or subset of A, B, C such as (1) A alone, (2) B alone, (3) C alone, (4) A with B, (5) A with C, (6) B with C, and (7) A with B and with C. As used herein, the phrase “at least one of A and B” is intended to refer to any combination or subset of A and B such as (1) at least one A, (2) at least one B, and (3) at least one A and at least one B. Similarly, the phrase “at least one of A or B” is intended to refer to any combination or subset of A and B such as (1) at least one A, (2) at least one B, and (3) at least one A and at least one B

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, inventive subject matter may lie in less than all features of a single disclosed example.

1. A system for assessing cognitive decline, the system comprising:
a sensor suite configured to be used in conjunction with a vehicle, the sensor suite comprising:
one or more sensors configured to sense one or more physiological signals of a driver while the driver grips the steering wheel and operates the vehicle,
one or more converters configured to convert the one or more sensed physiological signals into physiological data representative of one or more physiological states of the driver while they operate the vehicle, and
an imaging device configured to capture image data representative of the vehicle within an environment of use; and
a computing device comprising one or more processors configured to:
receive the physiological data and the image data from the sensor suite, and
determine one or more biomarkers representative of cognitive decline based upon the physiological data and the image data.

2. The system of claim 1, wherein the one or more sensors comprise at least one of an electrodermal activity sensor, a heart rate sensor, an electromyography sensor, or a gripping force sensor.

3. The system of claim 1:
wherein the sensor suite comprises a flexible elongated strip comprising at least a first layer and a second layer;
wherein the first layer comprises a pair of interdigitated electrodes configured to implement an electrodermal activity sensor; and
wherein the second layer is configured to implement a force sensor, the second layer comprising:
a first electrode layer,
a second electrode layer, and
a force-sensitive layer between the first electrode layer and the second electrode layer, the force-sensitive layer having an electrical resistance that varies with a force applied to the force sensing layer by the first and second electrode layers in response to a force exerted on the steering wheel by one or more of the driver's hands.

4. The system of claim 3, wherein the first layer further comprises a reference electrode configured to, in conjunction with at least one of the pair of interdigitated electrodes, to implement an electromyography sensor.

5. The system of claim 3, wherein the force sensing layer comprises a piezoresistive material.

6. The system of claim 3, wherein at least one of the first and second electrode layers is at least partially separated from the force sensing layer by an air gap.

7. The system of claim 1, wherein the one or more sensors are implemented on one or more elongated flexible substrates adapted to be secured to the steering wheel.

8. The system of claim 7, wherein the one or more elongated flexible substrates are integrated into a steering wheel sleeve or cover adapted to be installed on the steering wheel to secure the one or more elongated flexible substrate to the steering wheel.

9. The system of claim 7, wherein the one or more elongated flexible substrates are adapted to be directly affixed to the steering wheel.

10. The system of claim 1, wherein the sensor suite is configured to:
sense one or more physiological signals of the driver while the driver grips the steering wheel to operate the vehicle;
convert the one or more sensed physiological signals into physiological data representative of one or more physiological states of the driver while they operate the vehicle;
capture image data representative of the vehicle within an environment of use; and
communicate the physiological data and the image data to a computing device.

11. The system of claim 1, wherein the one or more processors are configured to determine the biomarkers by:
determining, based upon the image data, one or more driving behaviors of the driver;
determining, based upon the physiological data, one or more physiological states of the driver that temporally overlap the one or more driving behaviors; and
determining the one or more biomarkers based upon the driving behaviors and the physiological states.

12. The system of claim 11, wherein the one or more processors are further configured to:
determine, based upon the image data, one or more driving conditions; and
determine the one or more biomarkers based upon the driving behaviors, the driving conditions, and the physiological states.

13. The system of claim 11, wherein the one or more driving behaviors include at least one of a driving lane deviation, an inappropriate inter-vehicle distance, or a missed stop sign event.

14. The system of claim 11, wherein the one or more driving behaviors are determined using one or more computer vision algorithms.

15. The system of claim 11, wherein the one or more driving behaviors are determined using one or more trained machine learning models.

16. The system of claim 1, wherein the one or more processors are configured to determine the biomarkers by:
forming one or more input vectors representing the physiological data and the image data; and
processing, with one or more trained machine learning models, the one or more input vectors to determine the one or more biomarkers.

17. The system of claim 16, wherein the computing device is remote from the vehicle, and wherein the sensor suite further comprises a communication interface configured to convey the physiological data and the image data to the computing device.

18. The system of claim 1, wherein the computing device is configured to:
receive physiological data representing one or more physiological states of a driver while they operate a vehicle, the physiological data captured using one or more sensors secured to a steering wheel used by the driver to operate the vehicle;
receive imaging device representative of the vehicle within an environment of use;
and determine one or more biomarkers representative of cognitive decline based upon the physiological data and the image data.

19. An apparatus, comprising:
one or more sensors adapted to be secured to a steering wheel of a vehicle, and configured to sense one or more physiological signals of a driver while the driver grips the steering wheel and operates the vehicle;
one or more converters configured to convert the one or more sensed physiological signals into physiological data representative of one or more physiological states of the driver while they operate the vehicle;
an imaging device configured to capture image data representative of the vehicle within an environment of use; and
a communication interface to transfer the physiological data and the image data to a computing device.

20.-27. (canceled)

28. A method, comprising:
sensing, with one or more sensors of a sensor suite adapted to be secured to a steering wheel of a vehicle, one or more physiological signals of a driver while the driver grips the steering wheel to operate the vehicle;
converting the one or more sensed physiological signals into physiological data representative of one or more physiological states of the driver while they operate the vehicle;
capturing image data representative of the vehicle within an environment of use; and
communicating the physiological data and the image data to a computing device.

29.-31. (canceled)

32. A method, comprising:
receiving physiological data representing one or more physiological states of a driver while they operate a vehicle, the physiological data captured using one or more sensors secured to a steering wheel used by the driver to operate the vehicle;
receiving imaging device representative of the vehicle within an environment of use; and
determining one or more biomarkers representative of cognitive decline based upon the physiological data and the image data.

33.-38. (canceled)

39. A tangible machine-readable storage medium storing instructions that, when executed by one or more processors, cause a machine to:
receive physiological data representing one or more physiological states of a driver while they operate a vehicle, the physiological data captured using one or more sensors secured to a steering wheel used by the driver to operate the vehicle;
receive imaging device representative of the vehicle within an environment of use; and
determine one or more biomarkers representative of cognitive decline based upon the physiological data and the image data.

40.-45. (canceled)
